(19) United States
(12) Patent Application Publication
(10) Pub. No.: US 2017/0176742 A1
(43) Pub. Date: Jun. 22, 2017

(54) OPTICAL FIBER SCANNER, ILLUMINATING DEVICE, AND OBSERVATION APPARATUS

(71) Applicant: OLYMPUS CORPORATION, Tokyo (JP)

(72) Inventors: Yasuaki KASAI, Saitama (JP); Hiroshi TSURUTA, Kanagawa (JP); Hirokazu YOKOTA, Tokyo (JP)

(73) Assignee: OLYMPUS CORPORATION, Tokyo (JP)

(21) Appl. No.: 15/450,547

(22) Filed: Mar. 6, 2017

Related U.S. Application Data
(63) Continuation of application No. PCT/JP2014/079708, filed on Nov. 10, 2014.

(51) Int. Cl. G02B 26/10; H04N 5/225; G02B 23/24; A61B 1/04

(52) U.S. Cl. CPC G02B 26/103; A61B 1/04; H04N 5/2256; G02B 23/2469; H04N 2005/2255

(57) ABSTRACT

Provided is an optical fiber scanner including: an illumination optical fiber; an annular elastic member formed of an elastic material having an engagement hole that engages with the illumination optical fiber; and a plurality of piezoelectric elements fixed to the annular elastic member, each piezoelectric element being polarized in a radial direction of the illumination optical fiber and applied an alternating voltage in a polarization direction so as to cause the illumination optical fiber to vibrate. The annular elastic member has a vibration transmitting section to a side of which the plurality of piezoelectric elements are bonded and transmits vibrations to the illumination optical fiber, and a fiber support section that is integrally molded together with the vibration transmitting section and that is capable of supporting the illumination optical fiber.

### CROSS-REFERENCE TO RELATED APPLICATIONS

This is a continuation of International Application PCT/JP2014/079708, with an international filing date of Nov. 10, 2014, which is hereby incorporated by reference herein in its entirety. This application claims the benefit of International Application PCT/JP2014/079708.

### TECHNICAL FIELD

The present invention relates to optical fiber scanners, illuminating devices, and observation apparatuses.

### BACKGROUND ART

A known optical fiber scanner in the related art emits illumination light while vibrating the distal end of an optical fiber at high speed by using piezoelectric elements so as to scan the illumination light over a subject (for example, see Patent Literature 1). In the optical fiber scanner described in Patent Literature 1, the optical fiber is supported by an elastic section formed of a substantially prismatic member having a plurality of piezoelectric elements bonded thereto. The elastic section is joined to a ring-shaped support section, and the two sections are retained in an endoscope frame.

### CITATION LIST

### Patent Literature

{PTL 1}
Japanese Unexamined Patent Application, Publication No. 2013-244045

### SUMMARY OF INVENTION

An object of present invention is to provide an optical fiber scanner, an illuminating device, and an observation apparatus that enable easy and highly-accurate assembly adjustment for centering an optical fiber relative to an illumination optical system.

### Solution to Problem

A first aspect of the present invention provides an optical fiber scanner including: an optical fiber that optically guides light and emits the light from a distal end thereof; an annular elastic member formed of an elastic material having an engagement hole that engages with a base side of the optical fiber relative to the distal end thereof; and a plurality of piezoelectric elements fixed to the annular elastic member, each piezoelectric element being polarized in a radial direction of the optical fiber and applied an alternating voltage in a polarization direction so as to cause the optical fiber to vibrate. The annular elastic member has a vibration transmitting section to a side surface of which the plurality of piezoelectric elements are bonded and transmits vibrations of the piezoelectric elements to the optical fiber, and a fiber support section that is integrally molded together with the vibration transmitting section and that is capable of supporting the optical fiber in a cantilevered manner at a position away from the piezoelectric elements on the vibration transmitting section toward the base side.

In the above aspect, the fiber support section may have, on an outer surface thereof, a groove that is capable of accommodating a wire connected to each piezoelectric element.

In the above aspect, the fiber support section may have a through-hole in which a wire connected to each piezoelectric element can be fitted.

A second aspect of the present invention provides an illuminating device including the aforementioned optical fiber scanner, a light source that generates light to be optically guided by the optical fiber, a focusing lens that focuses the light emitted from the optical fiber, and an outer casing that accommodates the focusing lens and the optical fiber scanner and that retains the fiber support section.

A third aspect of the present invention provides an observation apparatus including the aforementioned illuminating device and a light detector that detects return light returning from a subject as a result of the subject being irradiated with light by the illuminating device.

### BRIEF DESCRIPTION OF DRAWINGS

FIG. 1 schematically illustrates the overall configuration of an endoscope apparatus according to an embodiment of the present invention.

FIG. 2 schematically illustrates the configuration of an optical fiber scanner in FIG. 1.

FIG. 3 is a cross-sectional view of a vibration transmitting section in an annular elastic member in FIG. 2, taken in the radial direction of an illumination optical fiber.

FIG. 4 is a cross-sectional view of a fiber support section in the annular elastic member in FIG. 2, taken in the radial direction.

FIG. 5 schematically illustrates the overall configuration of an optical fiber scanner according to a first modification of the embodiment of the present invention.

FIG. 6 is a cross-sectional view of a vibration transmitting section in an annular elastic member in FIG. 5, taken in the radial direction.

FIG. 7 is a cross-sectional view of a fiber support section in the annular elastic member in FIG. 5, taken in the radial direction.

### DESCRIPTION OF EMBODIMENTS

An optical fiber scanner, an illuminating device, and an observation apparatus according to an embodiment of the present invention will be described below with reference to the drawings.

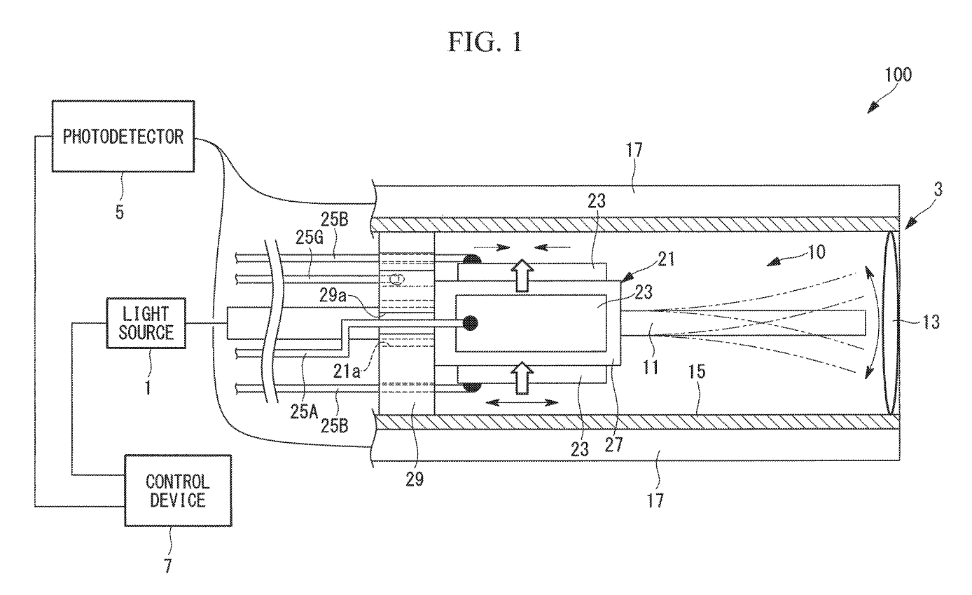

As shown in FIG. 1, an endoscope apparatus (observation apparatus) 100 according to this embodiment includes a light source 1 that generates illumination light, an illuminating device 3 that radiates the illumination light onto a subject (not shown), a photodetector (light detector) 5 that detects return light, such as reflected light or fluorescence, returning from the subject as a result of the subject being irradiated with the illumination light, and a control device 7 that controls, for example, the illuminating device 3 and the photodetector 5.

The illuminating device 3 includes an optical fiber scanner 10 having an illumination optical fiber 11 that optically guides the illumination light emitted from the light source 1 and emits the illumination light from the distal end thereof, a focusing lens 13 that focuses the illumination light emitted from the illumination optical fiber 11, a narrow tubular outer casing 15 that accommodates the optical fiber scanner 10 and the focusing lens 13 therein, and a plurality of detection optical fibers 17 that are disposed on the outer peripheral surface of the outer casing 15 and that optically guide the return light from the subject to the photodetector 5.

The light source 1 and the photodetector 5 are disposed at the base end of the optical fiber scanner 10.

The control device 7 includes a CPU (not shown) that controls the illuminating device 3 and the photodetector 5, and also includes a memory that stores, for example, a program for actuating the CPU and various kinds of signals to be input to the CPU.

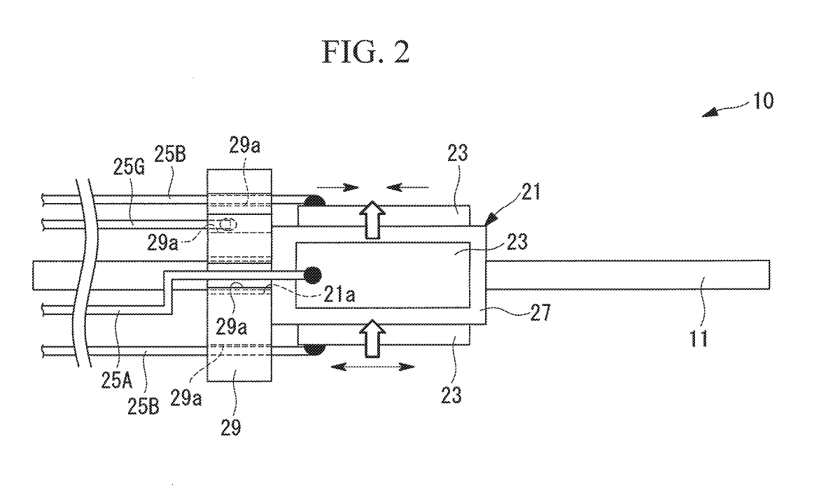

As shown in FIG. 2, the optical fiber scanner 10 includes the illumination optical fiber 11 (optical fiber), such as a multi-mode fiber or a single-mode fiber, an annular elastic member 21 that engages with the base side of the illumination optical fiber 11 relative to the distal end thereof and that is formed of an elastic material, four piezoelectric elements 23 fixed to the annular elastic member 21, and a driving lead wire (GND) 25G and four lead wires 25A and 25B.

As shown in FIGS. 1 and 2, the illumination optical fiber 11 is formed of a narrow glass material and is disposed in the longitudinal direction of the outer casing 15. With regard to the illumination optical fiber 11, one end thereof extends outward from the base end of the outer casing 15, whereas the other end thereof is disposed near the distal end inside the outer casing 15.

The annular elastic member 21 is formed by integral molding using nickel and is constituted of a narrow, tubular vibration transmitting section 27 and an annular fiber support section 29 having a diameter larger than that of the vibration transmitting section 27. The annular elastic member 21 is disposed such that the vibration transmitting section 27 thereof is oriented toward the distal end of the illumination optical fiber 11.

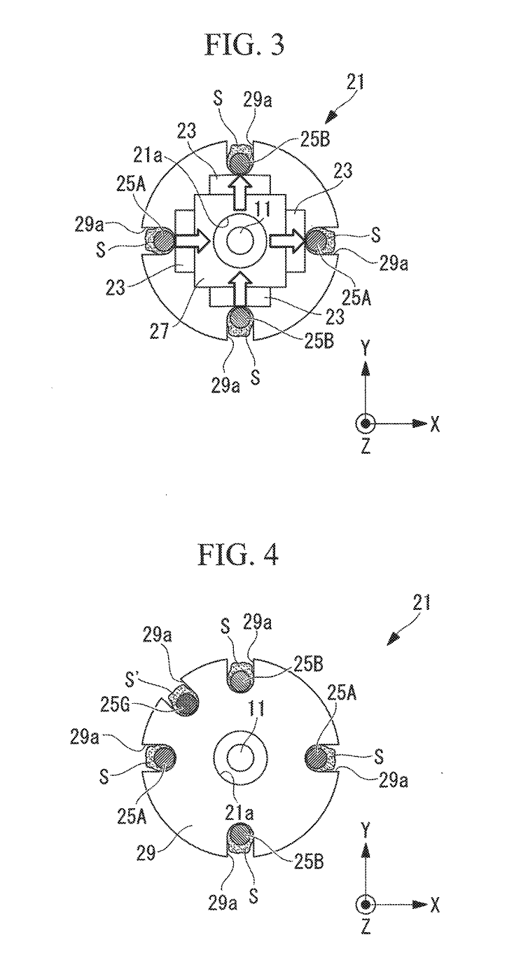

As shown in FIGS. 3 and 4, the vibration transmitting section 27 and the fiber support section 29 have an engagement hole 21a in which the illumination optical fiber 11 is fitted. In the engagement hole 21a, the engaged illumination optical fiber 11 is adhered thereto by using a conductive epoxy-based adhesive applied to the outer peripheral surface of the illumination optical fiber 11.

As shown in FIG. 3, the vibration transmitting section 27 has a substantially quadratic prism shape and has the piezoelectric elements 23 respectively bonded to the four side surfaces thereof. The vibration transmitting section 27 transmits vibrations occurring in the piezoelectric elements 23 to the illumination optical fiber 11.

The fiber support section 29 is ring-shaped, and the outer peripheral surface thereof is adhered to the inner wall of the outer casing 15 by using a conductive epoxy-based adhesive. The fiber support section 29 supports the illumination optical fiber 11 in a cantilevered manner at a position away from the piezoelectric elements 23 on the vibration transmitting section 27 toward the base end. Thus, the fiber support section 29 suppresses vibrations of the illumination optical fiber 11 occurring in the radial direction at this position. Supposing that the vibrations escape toward the base end of the illumination optical fiber 11 from the piezoelectric elements 23, the vibrations returning in a changed shape due to them receiving a certain effect can still be suppressed. Therefore, the fiber support section 29 can prevent the vibrating shape of the piezoelectric elements 23 and the vibrations of the illumination optical fiber 11 from becoming unstable.

The fiber support section 29 is electrically joined to electrodes on the back surfaces of the four piezoelectric elements 23 so as to function as a common ground (GND) when the piezoelectric elements 23 are driven. The fiber support section 29 is joined to the lead wire 25G. Moreover, as shown in FIGS. 3 and 4, the fiber support section 29 has, on the outer peripheral surface thereof, five grooves 29a that are recessed in the radial direction so as to be capable of accommodating the lead wire 25G and the four lead wires 25A and 25B therein.

The grooves 29a are circumferentially spaced apart from one another on the outer peripheral surface of the fiber support section 29 and extend parallel to the central axis. Therefore, the accommodated lead wires 25A and 25B can be connected to the piezoelectric elements 23 without making them longer than necessary. The grooves 29a have a depth with which the lead wires 25A, 25B, and 25G can be substantially entirely accommodated therein so that an epoxy-based adhesive (reference sign S in FIGS. 3 and 4) used for fixing the lead wires 25A and 25B in the corresponding grooves 29a and a conductive epoxy-based adhesive (reference sign S′ in FIG. 4) used for fixing the lead wire 25G in the corresponding groove 29a do not protrude therefrom. Consequently, the fiber support section 29 can be accurately engaged with the outer casing 15.

The annular elastic member 21 having this shape may be obtained by forming the vibration transmitting section 27 and the fiber support section 29 by performing wire electrical discharge machining on, for example, a pipe material having the same dimensions as the fiber support section 29 and having the engagement hole 21a formed therein. By similarly performing wire electrical discharge machining, the grooves 29a may be formed in the fiber support section 29. Although nickel is described as an example of the material of the annular elastic member 21, an alternative material that can transmit vibrations to the illumination optical fiber 11 and that can electrically function as a common ground (GND) may be used.

The piezoelectric elements 23 are each composed of a piezoelectric ceramic material, such as lead zirconate titanate (PZT), and have a narrow plate-like shape. Furthermore, each piezoelectric element 23 is given a positive electrode treatment on the front surface thereof and a negative electrode treatment on the back surface thereof so as to be polarized from the positive electrode toward the negative electrode, that is, in the thickness direction.

As shown in FIG. 2, the four piezoelectric elements 23 are disposed on the respective side surfaces of the vibration transmitting section 27 of the annular elastic member 21 at identical positions in the longitudinal direction of the illumination optical fiber 11. It is desirable that each piezoelectric element 23 and the fiber support section 29 be separated from each other by at least a gap that prevents interference with expansion and contraction of the piezoelectric element 23 in a direction intersecting the polarization direction thereof. Accordingly, expansion and contraction of the piezoelectric element 23 in the longitudinal direction of the illumination optical fiber 11 are not interfered with by the fiber support section 29.

As indicated by arrows denoting the directions of polarization in FIG. 3, each pair of piezoelectric elements 23 facing each other in the radial direction of the illumination optical fiber 11 is disposed such that the directions of polarization thereof are identical to each other. Moreover, by using a conductive epoxy-based adhesive, the lead wires 25A serving as A-phase lead wires are joined to the electrode surfaces of one of the pairs of piezoelectric elements 23, and the lead wires 25B serving as B-phase lead wires are joined to the electrode surfaces of the other pair of piezoelectric elements 23.

When an alternating voltage is applied in the polarization direction to the piezoelectric elements 23 via the lead wires 25A and 25B, vibrations (transverse effect) that cause the piezoelectric elements 23 to expand and contract in the direction orthogonal to the polarization direction occur. Moreover, the expansion and contraction occur in a manner such that when one of the piezoelectric elements 23 in each pair contracts, the other one expands concurrently therewith. Consequently, the piezoelectric elements 23 in each pair transmit the vibrations to the illumination optical fiber 11 via the vibration transmitting section 27 so as to cause the distal end of the illumination optical fiber 11 to vibrate in the direction intersecting the longitudinal direction.

As shown in FIG. 4, the lead wire 25G is accommodated in one of the grooves 29a in the fiber support section 29, and one end thereof is joined to the groove 29a by using the conductive epoxy-based adhesive S′. The lead wires 25A and 25B connected to the piezoelectric elements 23 are accommodated in the remaining grooves 29a in the fiber support section 29 and are fixed to the grooves 29a by using the epoxy-based adhesive S.

As shown in FIG. 1, the detection optical fibers 17 are each formed of a narrow glass material and are disposed in the longitudinal direction on the outer peripheral surface of the outer casing 15. These detection optical fibers 17 are spaced apart from one another in the circumferential direction of the outer casing 15. With regard to each detection optical fiber 17, one end thereof is disposed at the distal end of the outer casing 15, whereas the other end thereof is connected to the photodetector 5.

In addition to controlling the illuminating device 3 and the photodetector 5, the control device 7 can generate image information by associating an intensity signal of return light detected by the photodetector 5 with information (scan-position information) related to the position of illumination light scanned by the optical fiber scanner 10.

The operation of the optical fiber scanner 10, the illuminating device 3, and the endoscope apparatus 100 having the above-described configuration will now be described.

In order to observe a subject by using the optical fiber scanner 10, the illuminating device 3, and the endoscope apparatus 100 according to this embodiment, the distal end of the outer casing 15 is first disposed facing the subject, and illumination light is generated from the light source 1. The illumination light emitted from the light source 1 is optically guided by the illumination optical fiber 11, is emitted from the distal end thereof, and is radiated onto the subject by the focusing lens 13.

When return light, such as reflected light or fluorescence, is generated in the subject as a result of the subject being irradiated with the illumination light, the return light is optically guided by the detection optical fibers 17 and is detected by the photodetector 5. Then, the control device 7 converts the return light into image information by associating an intensity signal of the return light emitted from the photodetector 5 with scan-position information of the optical fiber scanner 10. Consequently, an image of the subject irradiated with the illumination light can be generated.

Next, an illumination-light scanning process performed by the optical fiber scanner 10 will be described.

In order to cause the optical fiber scanner 10 to scan the illumination light, a bending resonance frequency of the illumination optical fiber 11 is first excited such that the central area of the fiber support section 29 of the annular elastic member 21 in the axial direction serves as a node and the distal end of the illumination optical fiber 11 serves as an antinode.

When an alternating voltage corresponding to the bending resonance frequency is applied to one of the pairs of piezoelectric elements 23 (referred to as “A-phase piezoelectric elements 23” hereinafter), the A-phase piezoelectric elements 23 vibrate. Then, the vibrations occurring in the A-phase piezoelectric elements 23 are transmitted to the illumination optical fiber 11 via the vibration transmitting section 27 of the annular elastic member 21, thus causing the distal end of the illumination optical fiber 11 to vibrate in one direction (for example, the X-axis (A-phase) direction in FIGS. 3 and 4) intersecting the longitudinal direction.

Likewise, when an alternating voltage corresponding to the bending resonance frequency is applied to the other pair of piezoelectric elements 23 (referred to as “B-phase piezoelectric elements 23” hereinafter), the B-phase piezoelectric elements 23 vibrate. Then, the vibrations occurring in the B-phase piezoelectric elements 23 are transmitted to the illumination optical fiber 11 via the vibration transmitting section 27 of the annular elastic member 21, thus causing the distal end of the illumination optical fiber 11 to vibrate in one direction (for example, the Y-axis (B-phase) direction in FIGS. 3 and 4) orthogonal to the X-axis direction.

By simultaneously causing the A-phase piezoelectric elements 23 to vibrate in the X-axis direction and the B-phase piezoelectric elements 23 to vibrate in the Y-axis direction and changing the phases of the alternating signals applied to the A-phase piezoelectric elements 23 and the B-phase piezoelectric elements 23 by π/2, the vibrations at the distal end of the illumination optical fiber 11 form a circular trajectory. By gradually adjusting (modulating) the magnitude of the alternating voltages applied to the A-phase piezoelectric elements 23 and the B-phase piezoelectric elements 23 in this state, the distal end of the illumination optical fiber 11 vibrates spirally. Consequently, the illumination light emitted from the distal end of the illumination optical fiber 11 can be scanned spirally over the subject.

In this case, in the optical fiber scanner 10 according to this embodiment, the annular elastic member 21 formed by integrally molding the vibration transmitting section 27 and the fiber support section 29 is used so that assembly variations occurring due to the effect of, for example, variations in the machining accuracy of the vibration transmitting section 27 and the fiber support section 29 can be suppressed. Consequently, when manufacturing the illuminating device 3, the central axis of the illumination optical fiber 11 can be readily aligned with the central axis of the focusing lens 13. This facilitates assembly adjustment for centering the illumination optical fiber 11 and the focusing lens 13, thereby achieving an improved yield rate.

By accommodating the lead wires 25A, 25B, and 25G in the five grooves 29a formed in the outer surface of the fiber support section 29 and fixing them by using the epoxy-based adhesive S and the conductive epoxy-based adhesive S′, the lead wires 25A, 25B, and 25G can be stably disposed, and the fiber support section 29 can be accurately engaged with the outer casing 15.

In the illuminating device 3 according to this embodiment, the illumination optical fiber 11 and the focusing lens 13 can be readily and accurately centered in accordance with this optical fiber scanner 10, thereby achieving improved performance. Therefore, the illumination light emitted from the light source 1 can be accurately scanned and can be radiated onto a desired position of the subject by the focusing lens 13.

With the endoscope apparatus 100 according to this embodiment, proper observation can be realized based on image information of a desired observation region of the subject obtained on the basis of an intensity signal of return light detected by the photodetector 5. In a case where the endoscope apparatus 100 is used for, for example, medical purposes, a high-precision scanned image can be obtained without being dependent on the environment within the body cavity. For example, even in the case of a small site within the biological body, proper observation can be performed without being affected much by various kinds of body motions, such as heartbeats, breathing, and peristalsis.

This embodiment can be modified as follows.

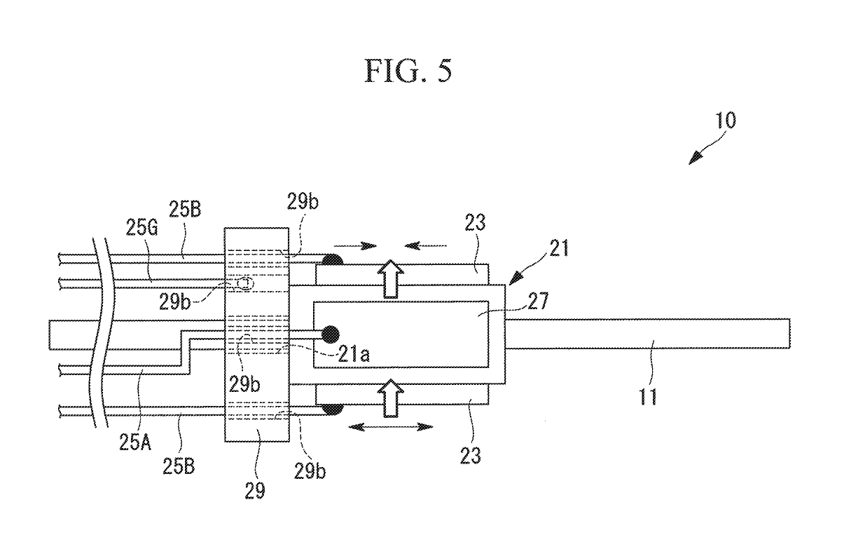

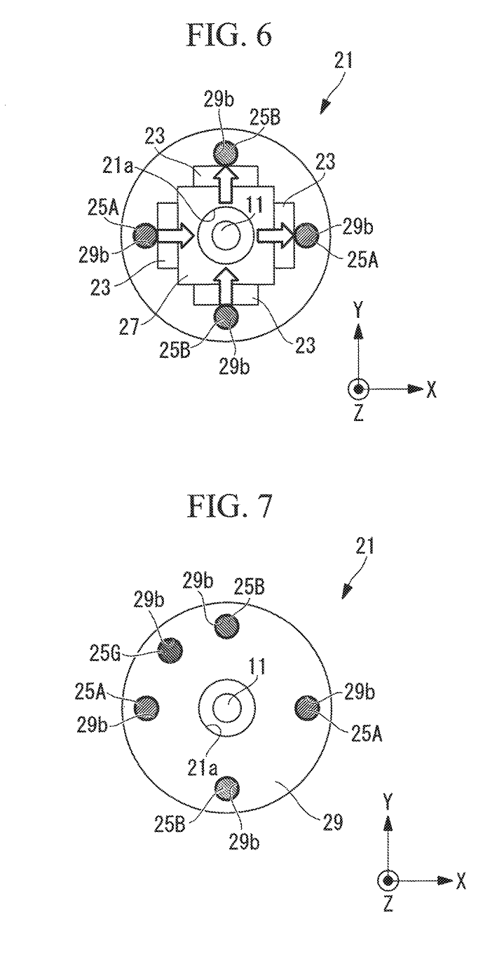

In this embodiment, the fiber support section 29 has, on the outer peripheral surface thereof, the grooves 29a that can accommodate the lead wires 25 therein. Alternatively, as a first modification, for example, the fiber support section 29 may have through-holes 29b in which the lead wires 25 connected to the piezoelectric elements 23 can be fitted, as shown in FIGS. 5, 6, and 7.

In this case, the lead wires 25A and 25B may be inserted through the corresponding through-holes 29b in the fiber support section 29 so as to be connected to the piezoelectric elements 23. Moreover, the lead wires 25A and 25B may be fixed to the through-holes 29b by using the epoxy-based adhesive S. Furthermore, the lead wire 25G may also be inserted through the corresponding through-hole 29b in the fiber support section 29, and one end thereof may be joined to the through-hole 29b by using the conductive epoxy-based adhesive S′. Consequently, the lead wires 25A and 25B can be stably disposed. This also facilitates the assembly adjustment for centering the illumination optical fiber 11 and the focusing lens 13 and for connecting the lead wires 25A and 25B to the piezoelectric elements 23.

By replacing the grooves 29a with the through-holes 29b, the epoxy-based adhesive S and the conductive epoxy-based adhesive S′ are less likely to protrude outward from the fiber support section 29, so that the fiber support section 29 can be accurately engaged with the outer casing 15. This configuration can achieve stability against shaking of the optical fiber scanner 10 other than vibrations and also against vibrations occurring outside the optical fiber scanner 10, such as a body motion of the subject being observed.

It is desirable that the through-holes 29b be parallel to the central axis of the fiber support section 29. Accordingly, the lead wires 25A, 25B, and 25G can be connected to the piezoelectric elements 23 without making them longer than necessary. Moreover, the through-holes 29b may be formed by, for example, drilling.

Although the embodiment of the present invention has been described above in detail with reference to the drawings, specific configurations are not limited to this embodiment and include, for example, design alterations so long as they do not deviate from the scope of the invention. For example, the present invention is not limited to the above-described embodiment and the modifications thereof. The present invention is not particularly limited and may be applied to embodiments obtained by appropriately combining the embodiment and the modifications thereof.

As a result, the following aspect is read by the above described embodiment of the present invention.

A first aspect of the present invention provides an optical fiber scanner including: an optical fiber that optically guides light and emits the light from a distal end thereof; an annular elastic member formed of an elastic material having an engagement hole that engages with a base side of the optical fiber relative to the distal end thereof; and a plurality of piezoelectric elements fixed to the annular elastic member, each piezoelectric element being polarized in a radial direction of the optical fiber and applied an alternating voltage in a polarization direction so as to cause the optical fiber to vibrate. The annular elastic member has a vibration transmitting section to a side surface of which the plurality of piezoelectric elements are bonded and transmits vibrations of the piezoelectric elements to the optical fiber, and a fiber support section that is integrally molded together with the vibration transmitting section and that is capable of supporting the optical fiber in a cantilevered manner at a position away from the piezoelectric elements on the vibration transmitting section toward the base side.

According to this aspect, when an alternating voltage is applied to each piezoelectric element in the polarization direction thereof, the piezoelectric element vibrates by expanding and contracting in the direction orthogonal to the polarization direction, that is, the longitudinal direction of the optical fiber, and the vibrations of the piezoelectric element are transmitted to the optical fiber via the vibration transmitting section of the annular elastic member. Furthermore, the fiber support section of the annular elastic member supports the optical fiber in a cantilevered manner so as to prevent the vibrations occurring in each piezoelectric element from escaping toward the base side of the optical fiber. Consequently, the distal end of the optical fiber is stably vibrated, so that the light emitted from the distal end of the optical fiber can be accurately scanned in accordance with the vibrations of the optical fiber.

In this case, the annular elastic member formed by integrally molding the vibration transmitting section and the fiber support section is used so that assembly variations occurring due to the effect of, for example, variations in the machining accuracy of the vibration transmitting section and the fiber support section can be suppressed. Consequently, the central axis of the optical fiber can be readily aligned with the central axis of an illumination optical system that radiates the light emitted from the optical fiber onto a subject. This facilitates assembly adjustment for centering the optical fiber and the illumination optical system, thereby achieving an improved yield rate.

In the above aspect, the fiber support section may have, on an outer surface thereof, a groove that is capable of accommodating a wire connected to each piezoelectric element.

With this configuration, the wire connected to each piezoelectric element is accommodated in the groove in the fiber support section and is fixed thereto by using, for example, an adhesive, so that the wire can be stably disposed. It is desirable that the groove be formed along the engagement hole, which engages with the optical fiber, in the fiber support section. Accordingly, the wire can be connected to each piezoelectric element without making it longer than necessary. Moreover, it is desirable that the groove have a depth with which the wire can be substantially entirely accommodated therein so that the adhesive does not protrude therefrom when the accommodated wire is fixed by using the adhesive. Accordingly, the fiber support section can be readily engaged with the outer casing.

In the above aspect, the fiber support section may have a through-hole in which a wire connected to each piezoelectric element can be fitted.

With this configuration, the wire is inserted through the through-hole in the fiber support section, is connected to each piezoelectric element, and is fixed to the through-hole by using, for example, an adhesive, so that the wire can be stably disposed. Moreover, the adhesive is less likely to protrude outward from the fiber support section, so that the fiber support section can be accurately engaged with the outer casing. It is desirable that the through-hole be formed along the engagement hole, which engages with the optical fiber, in the fiber support section. Accordingly, the wire can be connected to each piezoelectric element without making it longer than necessary.

A second aspect of the present invention provides an illuminating device including the aforementioned optical fiber scanner, a light source that generates light to be optically guided by the optical fiber, a focusing lens that focuses the light emitted from the optical fiber, and an outer casing that accommodates the focusing lens and the optical fiber scanner and that retains the fiber support section.

According to this aspect, the optical fiber and the focusing lens can be readily and accurately centered, thereby achieving improved performance of the illuminating device. Therefore, the light emitted from the light source can be accurately scanned and be radiated onto a desired position of a subject by the focusing lens.

A third aspect of the present invention provides an observation apparatus including the aforementioned illuminating device and a light detector that detects return light returning from a subject as a result of the subject being irradiated with light by the illuminating device.

According to this aspect, light is accurately scanned at a desired position of the subject by the illuminating device so that return light returning from the subject is detected by the light detector. Therefore, proper observation can be realized based on image information of a desired observation region of the subject obtained on the basis of an intensity signal of the return light detected by the light detector.

### REFERENCE SIGNS LIST

1 light source
3 illuminating device
5 photodetector (light detector)
10 optical fiber scanner
11 illumination optical fiber (optical fiber)
13 focusing lens
15 outer casing
21 annular elastic member
21a engagement hole
23 piezoelectric element
27 vibration transmitting section
29 fiber support section
29a groove
29b through-hole
100 endoscope apparatus (observation apparatus)

1. An optical fiber scanner comprising:
an optical fiber that optically guides light and emits the light from a distal end thereof;
an annular elastic member formed of an elastic material having an engagement hole that engages with a base side of the optical fiber relative to the distal end thereof; and
a plurality of piezoelectric elements fixed to the annular elastic member, each piezoelectric element being polarized in a radial direction of the optical fiber and applied an alternating voltage in a polarization direction so as to cause the optical fiber to vibrate,
wherein the annular elastic member has a vibration transmitting section to a side surface of which the plurality of piezoelectric elements are bonded and transmits vibrations of the piezoelectric elements to the optical fiber, and a fiber support section that is integrally molded together with the vibration transmitting section and that is capable of supporting the optical fiber in a cantilevered manner at a position away from the piezoelectric elements on the vibration transmitting section toward the base side.

2. The optical fiber scanner according to claim 1,
wherein the fiber support section has, on an outer surface thereof, a groove that is capable of accommodating a wire connected to each piezoelectric element.

3. The optical fiber scanner according to claim 1,
wherein the fiber support section has a through-hole in which a wire connected to each piezoelectric element can be fitted.

4. An illuminating device comprising:
the optical fiber scanner according to claim 1;
a light source that generates light to be optically guided by the optical fiber;
a focusing lens that focuses the light emitted from the optical fiber; and
an outer casing that accommodates the focusing lens and the optical fiber scanner and that retains the fiber support section.

5. An observation apparatus comprising:
the illuminating device according to claim 4; and
a light detector that detects return light returning from a subject as a result of the subject being irradiated with light by the illuminating device.
